(19) United States
(12) Plant Patent Application Publication
(10) Pub. No.: US 2019/0320569 P1
(43) Pub. Date: Oct. 17, 2019

(54) Ipomoea Batatas Plant Named 'Fukumurasaki'

(50) ;

(71) Applicant: National Agriculture and Food Research Organization, Tsukuba-si (JP)

(72) Inventors: Yumi KAI, Tsukuba-si (JP); Akira KOBAYAHSI, Tsukuba-si (JP); Takeo SAKAIGAICHI, Tsukuba-si (JP); Keisuke SUEMATSU, Tsukuba-si (JP); Yasuhiro TAKAHATA, Tsukuba-si (JP); Masaru YOSHINAGA, Tsukuba-si (JP); Kenji KATAYAMA, Tsukuba-si (JP); Tetsufumi SAKAI, Tsukuba-si (JP); Toshiro FUJITA, Tsukuba-si (JP)

(21) Appl. No.: 16/501,452

(22) Filed: Apr. 16, 2019

(30) Foreign Application Priority Data
Apr. 16, 2018 (JP) 33005

(51) Int. Cl. A01H 6/00

(52) U.S. Cl. PLT/258

(57) ABSTRACT

‘Fukumurasaki’ is a new variety of sweet potato plant bred by crossbreeding variety ‘Kyukei 255’ and ‘Purple Sweet Lord’. The plant may be used, e.g., for cultivation of root tubers for consumption. The steamed flesh of ‘Fukumurasaki’ has a purple color, high sugar content, medium to viscous flesh quality, and excellent taste.

Plant name and variety denomination: Latin name of the genus and species of the plant claimed: Ipomoea batatas. The claimed plant is a hybridization of different species. Common names of the claimed plant's species: sweet potato.

Variety denomination: ‘Fukumurasaki’.

### BACKGROUND

Ipomoea batatas, commonly called sweet potato or sweet potato vine, is a dicotyledonous plant that belongs to the bindweed or morning glory family, Convolvulaceae. It is an herbaceous perennial that is cultivated for its edible root tubers. Ipomoea batatas is native to tropical regions of the Americas.

### BRIEF SUMMARY

‘Fukumurasaki’ is a new variety of Ipomoea batatas bred by crossbreeding variety ‘Kyukei 255’ as the female parent and ‘Purple Sweet Lord’ as the male parent. The plant may be used, e.g., for cultivation of root tubers for consumption.

‘Fukumurasaki’ is a purple-colored lineage containing anthocyanins in its storage root. Although the yield of the good quality potatoes is inferior to that of ‘Kokei No. 14’ and ‘Purple Sweet Lord’, the steamed flesh of ‘Fukumurasaki’ has high sugar content, medium to viscous flesh quality, and excellent taste.

The flesh quality of the ‘Purple Sweet Lord’ is powdery and the sweetness is insufficient compared to the yellow flesh sweet potato. ‘Fukumurasaki’ has a lower yield of good quality potatoes than ‘Kokei No. 14’ and ‘Purple Sweet Lord,’ but has a medium to viscous flesh quality, and taste of the steamed flesh is medium to excellent. Because of the moderate resistance of ‘Fukumurasaki’ to sweet potato nematodes, cultivation in nematodes-rich areas should be avoided or controlled with pesticide or other measures.

### BRIEF DESCRIPTION OF THE DRAWINGS

FIG. 1A is a photograph of ‘Fukumurasaki’ (left) leaves compared to ‘Kokei No. 14’ (right).

FIG. 1B is a photograph of ‘Fukumurasaki’ (left) leaves compared to ‘Purple Sweet Lord’ (right).

FIG. 2A is a photograph of ‘Fukumurasaki’ (left) root tuber and cross-section compared to ‘Kokei No. 14’ (right).

FIG. 2B is a photograph of ‘Fukumurasaki’ (left) root tuber and cross-section compared to ‘Purple Sweet Lord’ (right).

FIG. 3 is a photograph of the cultivation zone of ‘Fukumurasaki’.

FIG. 4 is a photograph of the whole plant body of Fukumurasaki’ (left) compared to ‘Purple Sweet Lord’ (right).

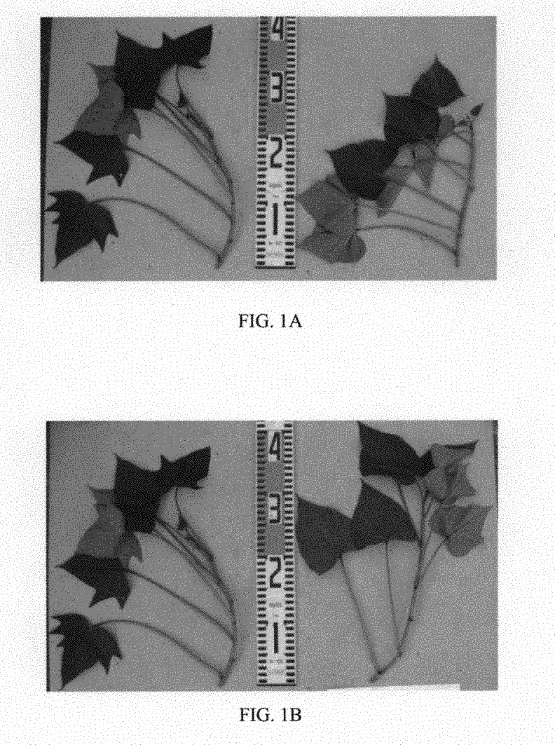

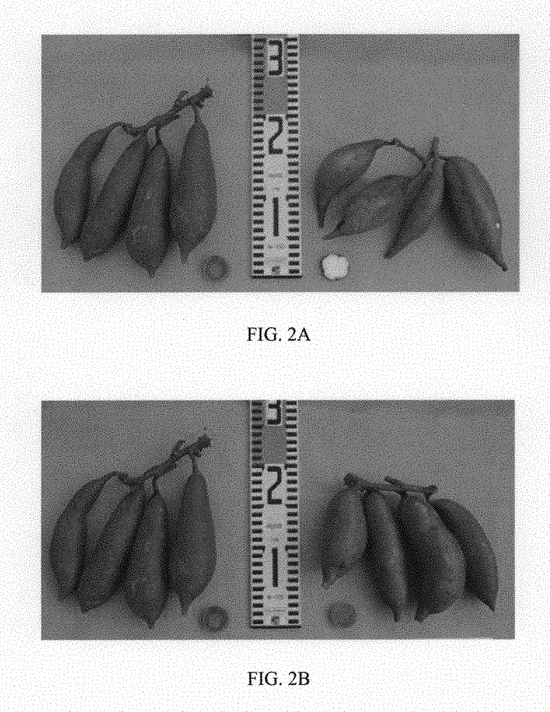

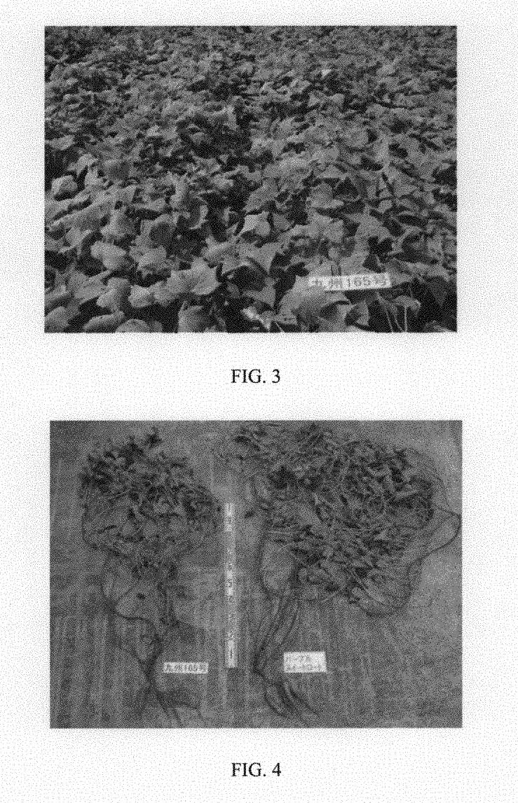

### DETAILED BOTANICAL DESCRIPTION OF THE PLANT

Variety: ‘Fukumurasaki’

Species of the plant claimed: Ipomoea batatas. The claimed plant is a hybridization of different varieties. The parental varieties are hybridization of different varieties.

Common names of the claimed plant's species include: sweet potato.

Characteristics of ‘Fukumurasaki’ provided herein were observed when the plant was cultivated in Okinawa and Satsuma, Japan. Descriptive terms used in Table 1 are consistent with the Japanese Examination Standard for Ipomoea batatas, available at

www.hinshu2.maff.go.jp/info/sinsakijun/kijun/1347.pdf, the entire contents of which are incorporated herein by reference.

Properties and characteristics of ‘Fukumurasaki’, compared to the most similar varieties, are described in Table 1.

TABLE 1
Properties and characteristics of ‘Fukumurasaki’

| | | | | Class Values of most | |
|---|---|---|---|---|---|
| | | Class | Remarks | similar varieties | |
| Charac- | | values of | (Measure- | | Purple |
| teristic | Charac- | candidate | ment | Kokei | Sweet |
| No. | teristics | variety | Value etc.) | No. 14 | Lord |
| 1 | Plant: growth | semi- | | spreading | spreading |
| | habit | upright | | | |
| 2 | Stem: length | short | 46.8 cm | medium | long |
| | of primary | | | (106.2 cm) | (157.4 cm) |
| | shoots | | | | |
| 3 | Stem: length | short | 2.6 cm | medium | long |
| | of internode | | | (5.1 cm) | (8.5 cm) |
| 4 | Stem: | large | 7.65 mm | medium to | medium to |
| | diameter | | | large | large |
| | of internode | | | (6.50 mm) | (6.67 mm) |
| 5 | Stem: | weak | | absent or | weak |
| | anthocyanin | | | very weak | |
| | coloration of | | | | |
| | internode | | | | |
| 6 | Stem: | weak | | absent or | weak |
| | anthocyanin | | | very weak | |
| | coloration | | | | |
| | of tip | | | | |
| 7 | Stem: | medium | | absent or | strong |
| | anthocyanin | | | very weak | |
| | coloration | | | | |
| | of node | | | | |
| 8 | Stem: | dense | | sparse | sparse |
| | pubescence | | | | |
| | of tip | | | | |
| 9 | Leaf blade: | large | 250.1 cm² | medium | medium to |
| | size | | | (160.9 cm²) | large |
| | | | | | (218.7 cm²) |
| 10 | Leaf blade: | five lobes | | absent | absent |
| | number of | | | | |
| | lobes | | | | |
| 11 | Only | — | | cordate | triangular |
| | varieties | | | | |
| | without | | | | |
| | leaf blade | | | | |
| | lobes: Leaf | | | | |
| | blade: shape | | | | |
| 12 | Only | moderate | | — | — |
| | varieties | | | | |
| | with leaf | | | | |
| | blade lobes: | | | | |
| | Leaf blade: | | | | |
| | depth of | | | | |
| | lobing | | | | |
| 13 | Excluding | green | | green | green |
| | ornamental | | | | |
| | variety: Leaf | | | | |
| | blade: color | | | | |
| | on upper side | | | | |
| | (excluding | | | | |
| | anthocyanin | | | | |
| | coloration) | | | | |
| 19 | Excluding | absent or | | absent or | absent or |
| | ornamental | very weak | | very weak | very weak |
| | variety: | | | | |
| | Leaf blade: | | | | |
| | anthocyanin | | | | |
| | coloration | | | | |
| | on upper side | | | | |
| 20 | Leaf blade: | absent or | | small | large |
| | extent of | very small | | | |
| | anthocyanin | | | | |
| | coloration | | | | |
| | on abaxial | | | | |
| | veins on | | | | |
| | lower side | | | | |
| 21 | Leaf blade: | very weak | | very weak | weak |
| | intensity of | | | | |
| | anthocyanin | | | | |
| | coloration | | | | |
| | on abaxial | | | | |
| | veins on | | | | |
| | lower side | | | | |
| 22 | Leaf blade: | absent or | | absent or | strong |
| | anthocyanin | very weak | | very weak | |
| | coloration of | | | | |
| | nectary | | | | |
| 23 | Young leaf | dark green | | light green | dark green |
| | blade: main | | | | |
| | color on | | | | |
| | upper side | | | | |
| 24 | Young leaf | medium | | light green | medium |
| | blade: main | green | | | green |
| | color on | | | | |
| | lower side | | | | |
| 25 | Petiole: | absent or | | absent or | absent or |
| | anthocyanin | very weak | | very weak | very weak |
| | coloration | | | | |
| 26 | Petiole: | medium | 24.4 cm | medium | medium to |
| | length | | | (22.4 cm) | long |
| | | | | | (26.7 cm) |
| 27 | Corolla: | — | | — | — |
| | diameter | | | | |
| 28 | Corolla: | — | | — | — |
| | color | | | | |
| 29 | Storage root: | ovate | | elliptic | oblong |
| | shape | | | | |
| 30 | Storage root: | moder- | 4.6 | moder- | medium |
| | ratio | ately | | ately | (2.9) |
| | length/width | elongated | | elongated | |
| | | | | (4.0) | |
| 31 | Storage root: | medium | 3.8 storage | medium | medium |
| | number | | roots/plant | (3.1 storage | (3.9 storage |
| | | | | roots/plant) | roots/plant) |
| 32 | Storage root: | medium | 142 g | medium | medium to |
| | mean | | | (146 g) | large |
| | weight | | | | (182 g) |
| 33 | Storage root: | thick | 0.072 | medium | medium |
| | thickness | | | (0.055) | (0.052) |
| | of cortex | | | | |
| | relative to | | | | |
| | overall | | | | |
| | diameter | | | | |
| 34 | Storage root: | purple red | | red | purple red |
| | main color | | | | |
| | of skin | | | | |
| 35 | Storage root: | absent | | absent | absent |
| | secondary | | | | |
| | color of skin | | | | |
| 36 | Storage root: | purple | | light beige | purple |
| | main color | | | | |
| | of flesh | | | | |
| 37 | Storage root: | medium | | medium | medium |
| | intensity of | | | | |
| | main color | | | | |
| | of flesh | | | | |
| 38 | Storage root: | white | | — | white |
| | secondary | | | | |
| | color of flesh | | | | |
| 39 | Storage root: | shallow | | medium | medium |
| | depth of eyes | | | | |
| 40 | Storage root: | purple | | light beige | light |
| | color of | | | | purple |
| | steamed flesh | | | | |
| 41 | Southern | moder- | | moder- | strong |
| | root-knot | ately | | ately | |
| | nematode | strong | | weak | |
| | resistance | | | | |

Cross-breeding was carried out in 2004 at the Sweet potato Breeding Laboratory of the Kyushu Okinawa Agricultural Research Center (currently the Sweet potato Breeding Group of the Kyushu Okinawa Agricultural Research Center) in Miyazaki prefucture, Japan. From 2005 onwards, selection and breeding were carried out at the Sweet potato Breeding Laboratory (currently the Sweet potato Breeding Group) in Japan. Based on the result of seedling individual selection test in 2005, a lineage was selected which showed excellent in appearance and root tuberization, and gave the lineage number of “Kyukei 04008-3”. Thereafter, they were used for the lineage selection preliminary test in 2006, the lineage selection test in 2007, and the productivity check preliminary test in 2008. As a result of examining various characteristics, we selected a lineage with excellent color of flesh and excellent characteristics of the steamed flesh, from “Kyukei 04008-3”, and gave the lineage number of “Kyukei 288”. And, since 2009, a productivity check test, a lineage adaptability test (Nagasaki Agricultural and Forestry Techinichal Development Center , Kagoshima Agricultural Research Center , Osumi Branch, Ehime Agriculture, Forestry, and Fisheries Research Center, etc.), a black rot resistance test (Nagasaki Agricultural and Forestry Techinichal Development Center), and a damping-off resistance test (Tokushima Agriculture, Forestry, and Fisheries Technology Support Center,) have been carried out, with the lineage number of “Kyukei 288”. Finally, ‘Fukumurasaki’ was selected by comprehensively examining the results of these tests.

### Morphological Characteristic

The plant growth habit in a field is the “semi-upright” type, and length of primary shoots and the length of internode of the stem are shorter and the diameter of internode is slightly larger than the ‘Kokei No. 14’ and the ‘Purple Sweet Lord’.

The coloration of ‘Fukumurasaki’ internode is “weak” and the coloration of node is “weak to medium”, the color on upper side of young leaf is “dark green” and the color on back side of young leaf is “green”, the leaf color is “green”, the leaf size is “large”, the number of lobes in leaf blade is “5”, and the leaf shape according to the old reference is “double-incision”.

The extent of the coloration on abaxial veins on lower side of the leaf is “absent or very small”, the intensity of the coloration of the same is “very weak”, and the coloration of the nectary is “absent or very weak”.

The strength of the storage root's neck and the position of the storage root (same as depth from the field surface to storage root) are “medium”, and the difficulty of digging (harvesting) the storage root (same as root tuber) is “medium”. The shape of storage root is “ovate” in the new standard and “long spindle” in the old standard.

Shape regularity of storage roots is “moderate”; size of storage roots is “medium”; size regularity of storage roots is “medium”; skin color of storage root is “purple red”; flesh color is “purple”; depth of eye in storage root is “shallow”; skin roughness of storage root is “slightly coarse”; grooves and ridges of storage root are “absent”; dehiscence of storage root is “little”; appearance is “medium”.

The shape of storage root is “ovate” or “long spindle”. Shape regularity of storage roots is “moderate”; size of storage roots is “medium”; size regularity of storage roots is “medium”; skin color of storage root is “purple red”; flesh color is “purple”; depth of eye in storage root is “shallow”; skin roughness of storage root is “slightly coarse”; grooves and ridges of storage root are “absent”; dehiscence of storage root is “little”; appearance is “medium”.

On average, the total weight of the good quality storage roots is 19% lower than that of ‘Kokei No. 14’. The mean weight of one good quality storage roots is lower than that of ‘Kokei No. 14’ and ‘Purple Sweet Lord’. The number of good quality storage roots per one plant is slightly larger than that of the ‘Kokei No. 14’ and equivalent to that of the ‘Purple Sweet Lord’. The percentage of dry matter content is about 5.6 points higher than that of ‘Kokei No. 14’.

At earlier harvest timing under cultivation with mulching sheet, the total weight of good quality storage roots of ‘Fukumurasaki’ is about 60% of that of ‘Kokei No. 14’. Under later-planting-timing cultivation without mulching sheet, the total weight of good quality storage roots is about 60% of that of ‘Kokei No. 14’. The mean weight of one good quality storage roots is lower than that of ‘Kokei No. 14’. The number of good quality storage roots per one plant is equivalent to that of ‘Kokei No. 14’. The percentage of dry matter content is about 5 points higher (at the earlier harvest timing), and about 7 points higher (under later-planting-timing cultivation) than those of ‘Kokei No. 14’.

### Ecological Characteristics

The timing (early or late) of sprouting of ‘Fukumurasaki’ from storage root, the timing regularity of sprouting from different storage roots (almost simultaneous or not), the speed of sprout elongation, and the number of sprouts are “medium”, and the sprouting trait (same as sprouting habit) is “medium”.

On average, the total weight of the good quality storage roots is 19% lower than that of ‘Kokei No. 14’. The mean weight of one good quality storage roots is lower than that of ‘Kokei No. 14’ and ‘Purple Sweet Lord’. The number of good quality storage roots per one ‘Fukumurasaki’ plant is slightly larger than that of the ‘Kokei No. 14’ and equivalent to that of the ‘Purple Sweet Lord’. The percentage of dry matter content is about 5.6 points higher than that of ‘Kokei No. 14’.

The resistance of ‘Fukumurasaki’ to sweet potato nematodes (M. incognita) is “medium” and the resistance to Minami root-lesion nematodas (P. coffeae) is “moderately strong”. The resistance to black rot disease is “medium to strong”. The resistance to damping-off is “slightly weak to slightly strong” , and the resistance to stem rot is “strong”. Storability of storage root is “medium”.

### Quality Characteristics and Processability

In standard cultivation, the steamed flesh color of the ‘Fukumurasaki’ storage root is “purple”, fibers in steamed flesh is “medium”, and the quality of flesh is “medium”. The taste of steamed or roasted storage root is “somewhat superior” and Brix is higher than ‘Kokei No. 14’ and ‘Purple Sweet Lord’.

### Plant Growth

The sprouting trait (same as sprouting habit) of ‘Fukumurasaki’ is “medium”. The plant growth habit in a field is the “semi-upright” type, and length of primary shoots and the length of internode of the stem are shorter and the diameter of internode is slightly larger than the ‘Kokei No. 14’ and the ‘Purple Sweet Lord’. The coloration of internode is “weak” and the coloration of node is “weak to medium”, the color on upper side of young leaf is “dark green” and the color on back side of young leaf is “green”, the leaf color is “green”, the leaf size is “large”, the number of lobes in leaf blade is “5”, and the leaf shape according to the old reference is “double-incision”. The extent of the coloration on abaxial veins on lower side of the leaf is “absent or very small”, the intensity of the coloration of the same is “very weak”, and the coloration of the nectary is “absent or very weak”.

‘Fukumurasaki’ was good in shape in Saitama Prefecture, and although the yield was poor, the taste (medium) was comparable to that of Beniazma. The yield was comparable to that of Beniazma in Ehime Prefecture, and the weight of storage root was slightly small, and the appearance quality of storage root was moderate, then over-all evaluation is X (inferior). Although the weight of storage root was small, the taste was slightly higher in Tokushima Prefecture, the sugar content was also high, then over-all evaluation is A (medium). Although the yield was small in Nagasaki Prefecture, the appearance and shape of storage root were excellent, size regularity of storage roots was excellent, and the taste of storage roots was good as a sweet potato with purple flesh, then over-all evaluation is A (medium). In Miyazaki Prefecture, storage roots were small and low in yield, and the flesh color of storage roots was thinner than normal, then over-all evaluation is X (inferior). In Kagoshima Prefecture, the yield was lower than that of ‘Purple Sweet Lord’, the rate of A products (good quality storage roots) was slightly higher than that of ‘Purple Sweet Lord’, but mean weight of one A product was light, and the appearance was slightly superior, and Brix (sugar content) was high, and the taste was good, then over-all evaluation is A (medium). The appearance was good in Okinawa Prefecture, and the taste was medium, and size of storage roots tended to be small, then over-all evaluation is A (medium).

### Other Characteristics

The steamed flesh color of ‘Fukumurasaki’ is “purple”, and the quality of flesh is “medium”. Both of the taste of steamed storage root and roasted storage root are “somewhat superior” and Brix is higher than ‘Kokei No. 14’ and ‘Purple Sweet Lord’.

Brix of ‘Fukumurasaki’ steamed storage root is higher than ‘Kokei No. 14’ and ‘Purple Sweet Lord’ in both the cultivation with transparent mulching sheet at earlier harvest timing and the later-planting-timing cultivation without mulching.

The taste of ‘Fukumurasaki’ steamed storage root is “medium” under the cultivation with transparent mulching sheet at earlier harvest timing, and “slightly higher” under the later-planting-timing cultivation without mulching.

In October, 2016, samples (storage roots) cultivated in the field in the city of Namegata were stored for about 70 days, and then each sample was roasted at 200° C. for 1 hour to prepare roasted samples (roasted sweet potatoes), and sensory evaluation by panelists was carried out. ‘Fukumurasaki’ was more viscous than ‘Purple Sweet Lord’ in terms of roasted flesh quality, superior in texture and sweetness, and ‘Fukumurasaki’ exceeded ‘Purple Sweet Lord’ in overall evaluation.

‘Fukumurasaki’ was grown in open culture. In Japan, planting occurs in the early part of May to the end part of May. In Japan harvesting time is the middle part of October to the end part of October.

It will be understood that the average size of the plant and storage root may vary with location, season, nutrition, irrigation, etc.

What is claimed is:

1. A new and distinct Ipomoea batatas plant named ‘Fukumurasaki’ as illustrated and described.
